(19) United States
(12) Patent Application Publication
(10) Pub. No.: US 2014/0018773 A1
(43) Pub. Date: Jan. 16, 2014

(54) GUIDE EXTENSION CATHETER

(71) Applicant: BOSTON SCIENTIFIC SCIMED, INC., MAPLE GROVE, MN (US)

(72) Inventors: HUISUN WANG, MAPLE GROVE, MN (US); JAMES M. ANDERSON, FRIDLEY, MN (US)

(21) Appl. No.: 13/941,198

(22) Filed: Jul. 12, 2013

Related U.S. Application Data
(60) Provisional application No. 61/671,501, filed on Jul. 13, 2012.

(51) Int. Cl. A61M 25/00

(52) U.S. Cl. CPC A61M 25/0021
USPC 604/510; 604/528

(57) ABSTRACT

Medical devices and methods for making and using medical devices are disclosed. An example medical device may include a guide extension catheter. The guide extension catheter may include a proximal member having a proximal outer diameter. A distal sheath member may be attached to the proximal member. The distal sheath member may have a proximal sheath portion and a distal sheath portion. The proximal sheath portion may have an outer diameter greater than the proximal outer diameter. The proximal sheath portion may have a first cross-sectional profile. The distal sheath portion may have a second cross-sectional profile different from the first cross-sectional profile.

### CROSS-REFERENCE TO RELATED APPLICATIONS

This application claims priority under 35 U.S.C. §119 to U.S. Provisional Application Ser. No. 61/671,501, filed Jul. 13, 2012, the entirety of which is incorporated herein by reference.

### TECHNICAL FIELD

The present disclosure pertains to medical devices, and methods for manufacturing medical devices. More particularly, the present disclosure pertains to elongated intracorporeal medical devices including a guide extension catheter.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a guide extension catheter. The guide extension catheter may include a proximal member having a proximal outer diameter. A distal sheath member may be attached to the proximal member. The distal sheath member may have a proximal sheath portion and a distal sheath portion. The proximal sheath portion may have an outer diameter greater than the proximal outer diameter. The proximal sheath portion may have a first cross-sectional profile. The distal sheath portion may have a second cross-sectional profile different from the first cross-sectional profile.

An example guide extension catheter system is also disclosed. The guide extension catheter system may include a guide catheter having an inner diameter and a guide extension catheter extending through the guide catheter. The guide extension catheter may include a proximal shaft and a distal sheath member attached to the proximal shaft. The distal sheath member may have proximal portion, a distal portion, and a tapered portion disposed between the proximal portion and the distal portion. The proximal portion of the distal sheath member may have an outer diameter that is configured to substantially fit within the inner diameter of the guide catheter.

Methods for accessing a coronary artery are also disclosed. An example method may include providing a guide catheter and advancing the guide catheter through a blood vessel to a position adjacent to an ostium of a coronary artery. The method may also include providing a guide extension catheter. The guide extension catheter may include a proximal shaft and a distal sheath member attached to the proximal shaft. The distal sheath member may have proximal portion, a distal portion, and a tapered portion disposed between the proximal portion and the distal portion. The proximal portion of the distal sheath member may have an outer diameter that is configured to substantially fit within the inner diameter of the guide catheter. The method may also include advancing the guide extension catheter through the guide catheter to a position where at least a portion of the distal sheath extends distally beyond a distal end of the guide catheter and into the coronary artery and advancing a treatment catheter through the guide catheter.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan view illustrating an example guide catheter advanced through the aorta to the ostium of a coronary artery;

FIG. 2 is a plan view illustrating an example guide extension catheter used in conjunction with a guide catheter;

FIG. 3 is a cross-sectional side view of an example guide extension catheter;

FIG. 4 is a cross-sectional side view of the example guide extension catheter and an example guide catheter;

FIG. 5 is a partial cross-sectional view of an example guide extension catheter;

FIG. 6A is a cross-sectional view taken through line 6-6 in FIG. 5;

FIG. 6B is an alternative cross-sectional view taken through line 6-6 in FIG. 5;

FIG. 7A is a cross-sectional view taken through line 7-7 in FIG. 5;

FIG. 7B is an alternative cross-sectional view taken through line 7-7 in FIG. 5;

FIG. 8 is a partial cross-sectional view of the example guide extension catheter illustrated in FIG. 5 and an example guide catheter;

FIG. 9 is a partial cross-sectional side view depicting an example medical device disposed in a blood vessel;

FIG. 10 is a transverse cross-sectional view of the example medical device illustrated in FIG. 9 disposed in a blood vessel;

FIG. 11 is a partial cross-sectional side view depicting an example guide extension catheter system disposed in a blood vessel;

FIG. 12 is a transverse cross-sectional view of the example guide extension catheter system illustrated in FIG. 11 disposed in a blood vessel;

FIG. 13 is an alternative transverse cross-sectional view of the example guide extension catheter system illustrated in FIG. 11 disposed in a blood vessel;

FIG. 14 is a side view of a portion of an example guide extension catheter;

FIG. 15 is a cross-sectional view taken through line 15-15 in FIG. 14;

FIG. 16 is a cross-sectional view taken through line 16-16 in FIG. 14;

FIG. 17 is a top view of a portion of an example guide extension catheter;

FIG. 18 is a side view of the example guide extension catheter illustrated in FIG. 17; and

FIG. 19 is a transverse cross-sectional view of the example guide extension catheter illustrated in FIGS. 17-18 disposed in a blood vessel.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

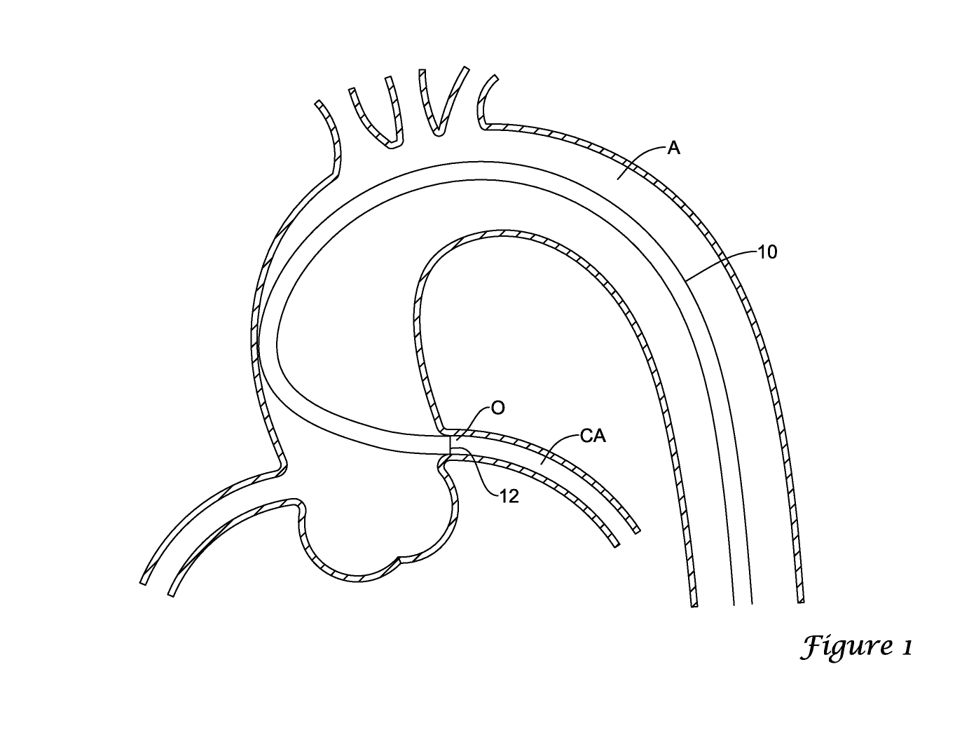

Minimally-invasive cardiac interventions such as percutaneous transluminal coronary angioplasty are widely utilized throughout the world. These procedures may include the use of a guide catheter. For example, a guide catheter 10 may be advanced through a blood vessel such as the aorta A to a position adjacent to the ostium O of a (e.g., left and/or right) coronary artery CA as illustrated in FIG. 1. When so positioned, a treatment catheter (e.g., balloon catheter, stent delivery system, etc.) may be advanced through guide catheter 10 and into the coronary artery CA to a target location where the treatment catheter may be used to perform the appropriate cardiac intervention. In order for the treatment catheter to efficiently reach the intended target location, maintaining the position of guide catheter 10 at the ostium O of the coronary artery CA may be desirable. For example, given that the heart may be beating during the intervention (and/or other factors), the guide catheter 10 may lose its positioning or otherwise be shifted so that it no longer is positioned to efficiently guide the treatment catheter to the coronary arteries. This may include a distal end 12 of guide catheter 10 being shifted away from the ostium O of the coronary artery CA. Because of the shift away from the ostium O, access to the coronary arteries CA may require repositioning of guide catheter 10 in order to bring the distal end 12 back into engagement with the ostium O of the coronary artery CA.

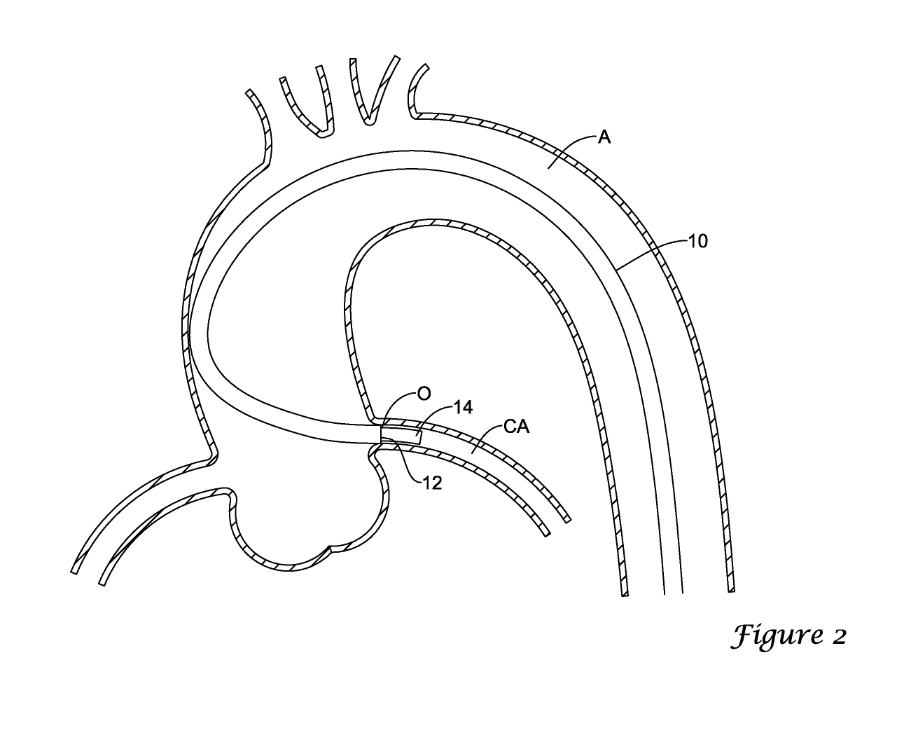

Disclosed herein are medical devices and methods for making and using medical devices that may improve access to the coronary arteries CA. For example, FIG. 2 illustrates a guide extension catheter 14 extending through guide catheter 10 and beyond distal end 12 of guide catheter 10 into the coronary artery CA. Because, for example, guide extension catheter 14 may extend beyond distal end 12 of guide catheter 10, guide extension catheter 14 may extend beyond the ostium O of the coronary artery CA and into a portion of the coronary artery CA. By extending beyond the ostium O, the extension catheter 14 may stabilize the positioning of guide catheter 10 and allow for improved access to the coronary artery CA for a number of cardiac interventions.

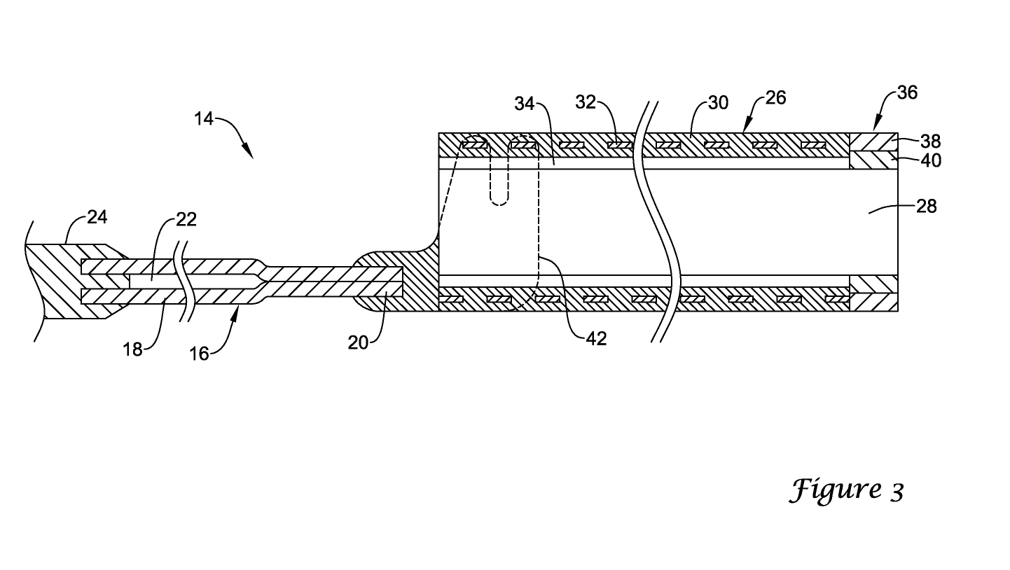

FIG. 3 is a cross-sectional side view of guide extension catheter 14. Here it can be seen that guide extension catheter 14 may include a proximal shaft or member 16. Proximal member 16 may include a proximal portion 18 and a distal or ribbon portion 20. Proximal portion 18 may have a lumen 22 defined therein. In some embodiments, lumen 22 extends along the entire length of proximal portion 18. In other embodiments, lumen 22 extends along only a portion of the length of proximal portion 18. In addition, proximal portion 18 may include both proximal and distal openings (e.g., positioned at the proximal and distal end of proximal portion 18) such that lumen 22 is “open” on both ends. Alternatively, one or both of the ends of proximal portion 18 may be closed or otherwise sealed. For example, the distal end of proximal portion 18 may be closed. In some of these and in other embodiments, proximal portion 18 may have an opening or port (not shown) formed in the wall of proximal portion 18 and spaced from the proximal and/or distal end of proximal portion 18. The port may or may not be in fluid communication with lumen 22. A hub 24 may be attached to proximal portion 18.

A distal sheath 26 may be attached to proximal member 16. Sheath 26 may have a lumen 28 formed therein. In general, lumen 28 (and/or the inner diameter of distal sheath 26) may be larger than lumen 22 (and/or the inner diameter of proximal portion 18) and may be larger than the outer diameter of proximal member 16. Accordingly, lumen 28 may be sufficiently large so as to allow a therapeutic catheter (e.g., balloon catheter, stent delivery system, etc.) to pass therethrough. For example, when guide extension catheter 14 is positioned within guide catheter 10, the therapeutic catheter may extend within guide catheter 10 alongside proximal member 16 and through lumen 28 of distal sheath 26.

Distal sheath 26 may include a body portion 30. In at least some embodiments, body portion 30 may include one or more polymers including any of those disclosed herein. This may include the use of polymers with a differing durometer along the length of body portion 30. For example, a more proximal section of body portion 30 may include a polymer with a higher durometer and a more distal section of body portion 30 may include a polymer with a lower durometer. Portions of all of the length of body portion may be loaded with or otherwise include a radiopaque material. Body portion 30 may also include a reinforcement member 32. The form of reinforcement member 32 may vary. For example, reinforcement member 32 may include a braid, coil, mesh, or the like.

An inner liner or layer 34 may be disposed along an inner surface of body portion 30. The form of liner 34 may vary. For example, liner 34 may be a lubricious liner or otherwise include a lubricious material such as polytetrafluoroethylene. A tip member 36 may be attached body portion 30, for example at a distal end of body portion 30. In some embodiments, tip member 36 may be a single layer of material. Alternatively, tip member may include an outer layer 38 and an inner layer 40. Outer layer 38 and inner layer 40 may be formed from the same material. In some of these embodiments, outer layer 38 and inner layer 40 may include the same polymeric material and each be loaded with the same or different radiopaque materials. For example, inner layer 40 may include a polyether block amide loaded with approximately 75-95% (e.g., about 90%) by weight tungsten and outer layer 38 may include a polyether block amide loaded with approximately 30-50% (e.g., 40%) by weight bismuth subcarbonate. These are just example. In other embodiments, outer layer 38 and inner layer 40 may be made from different materials.

Distal sheath 26 may be attached to ribbon portion 20 of proximal member 16. The arrangement and/or configuration of the attachment between ribbon portion 20 and distal sheath 26 may vary. For example, distal sheath 26 may have an opening or lumen formed in tube wall thereof and ribbon portion 20 may be disposed within the opening. This may include necking, skiving, or pinching down ribbon portion 20 and inserting the necked down portion into the opening. In some embodiments, inserting ribbon portion 20 into the opening may secure proximal member 16 to distal sheath 26 via a mechanical bond. In some of these and in other embodiments, additional and/or alternative bonding may be utilized including those bonding mechanisms commonly used for medical devices (e.g., adhesive bonding, welding, thermal bonding, brazing, etc.). Other attachment mechanisms are also contemplated for attaching proximal member 16 to distal sheath 26 including direct bonding (e.g., adhesive bonding, thermal bonding, welding, brazing, etc.), bonding that is facilitated by a third component such as a metal or polymer collar 42 that may be bonded between the ribbon portion 20 and distal sheath 26.

Guide extension catheter 14 may also include a number of coatings that may, for example, reduce friction. For example, proximal member 16 may have an inner and/or outer coating that includes a hydrophilic polymer that may reduce friction during tracking. An example coating may include BAYER CL-100, BIOSLIDE, NG-HPC, SLIP COAT, MDX, or the like. These are just examples. Other materials are contemplated including those disclosed herein.

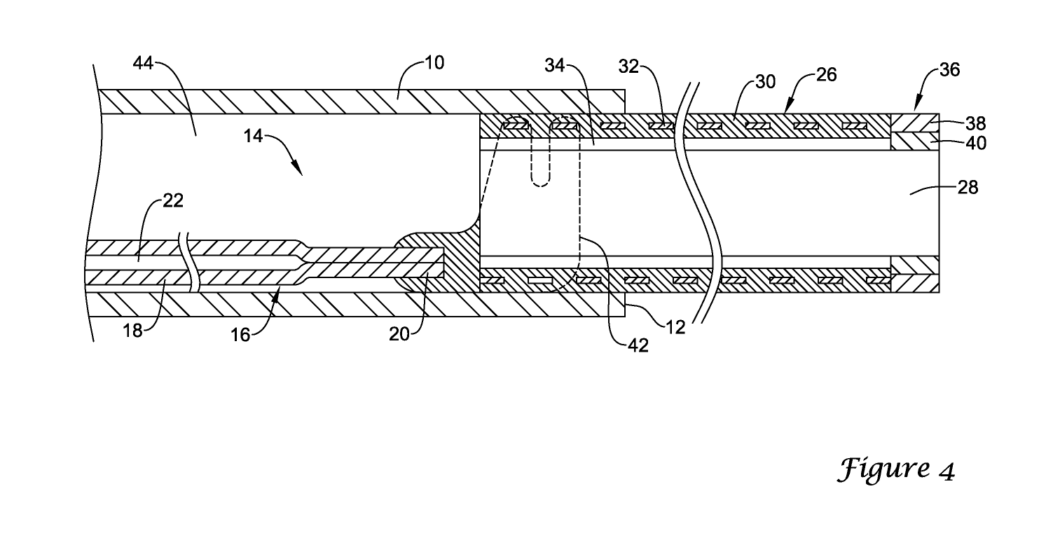

FIG. 4 illustrates guide extension catheter 14 disposed within guide catheter 10 (e.g., disposed within a lumen 44 defined within guide catheter 10). As shown, distal sheath 26 may be arranged to extend distally out from distal end 12 of guide catheter 10. When so arranged, distal sheath 26 may engage the ostium O and/or extend within a portion of the coronary artery CA to help maintain the position of guide catheter 10 and improve access to the coronary artery CA. Proximal member 16 may be designed to be sufficiently small (while still being sufficiently sized and configured for pushability) so as to take up relatively little space within the interior or lumen 44 of guide catheter 10.

Accordingly, the use of guide extension catheter 14 allows for a therapeutic catheter or medical device to be advanced through guide catheter 10 in order to reach the desired target location for the intervention. In some embodiments, proximal member 16 may contact the inner wall surface of guide catheter 10, which may provide even more space. When designing guide extension catheters like guide extension catheter 14, it may be desirable for the distal portion (e.g., distal sheath 26) to have an inner diameter sufficiently large for a therapeutic medical device to extend therethrough. Indeed, it may be desirable for the inner diameter of distal sheath 26 to closely approximate the outer diameter of the therapeutic medical device, while still allowing for the therapeutic medical device to easily be advancing through distal sheath 26. In addition, it may also be desirable for distal sheath 26 to have an outer diameter that approximates the inner diameter of guide catheter 10. A relatively close fit between the inner diameter of the distal sheath 26 and the therapeutic medical device as well as a relatively close fit between the outer diameter of distal sheath 26 and guide catheter 10 may remove excess open spaces between these structures and/or otherwise form a partially “sealed” arrangement between these structures. The sealed arrangement may aid in preventing contrast media that is infused into guide catheter 10 from simply exiting the distal end 12 of guide catheter 10. Due to the size differences between some guide catheters and therapeutic medical devices, a need exists for guide extension catheters that can provide the structural features needed to achieve a desirable close fit between inner diameter of the distal sheath 26 and the therapeutic medical device as well as a relatively close fit between the outer diameter of distal sheath 26 and guide catheter 10.

In addition, the relatively blunt distal end 12 of guide catheter 10 may also have a tendency to be present challenges for navigating guide catheter 10 through the anatomy. For example, the generally rounded distal end of typical guide catheters and other devices may catch on partial occlusions, total occlusions, calcified lesions, and the like. The same may also be true of some guide extension catheters and/or catheter systems. A need exists for guide extension catheters with improved crossing abilities for crossing, for example, partial occlusions, total occlusions, calcified lesions, and the like.

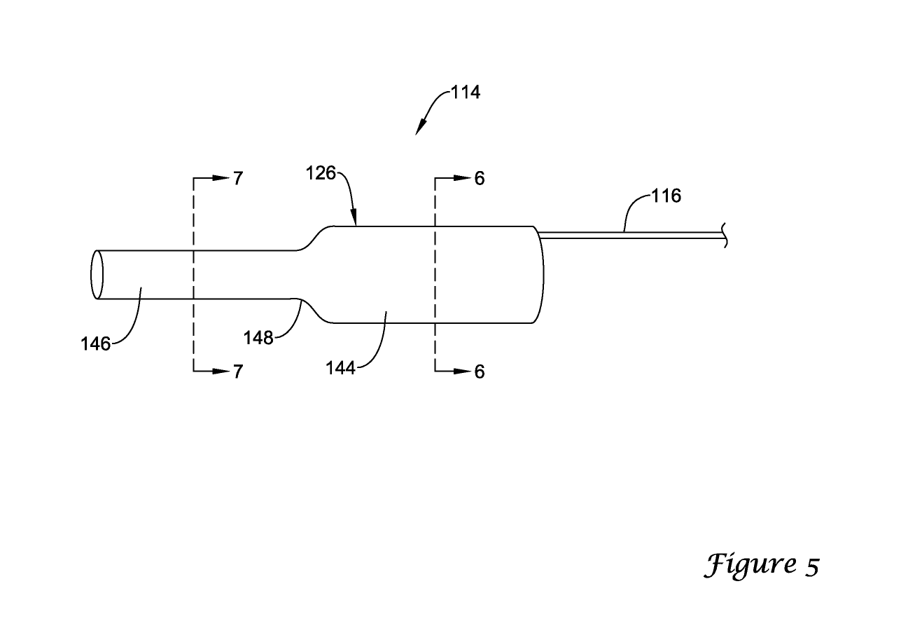

FIG. 5 illustrate an example guide extension catheter 114 that may be similar in form and function to other guide extension catheters disclosed herein. Guide extension catheter 114 includes one or more structural features the aid in forming a tighter seal or closer fit with one or more medical devices associated therewith such as guide catheter 10 and/or a therapeutic medical device. In addition, guide extension catheter 114 is designed to have improved crossing abilities for crossing, for example, partial occlusions, total occlusions, calcified lesions, and the like.

Guide extension catheter 114 may include proximal member 116 and distal sheath 126. The structures are shown schematically. It can be appreciated that the form and/or structural configuration of proximal member 116 and/or distal sheath 126 may resemble other proximal members and distal sheaths (e.g., proximal member 16 and distal sheath 26) disclosed herein. Distal sheath 126 may include a proximal portion 144, a distal portion 146, and a transition portion 148 disposed between proximal portion 144 and distal portion 146.

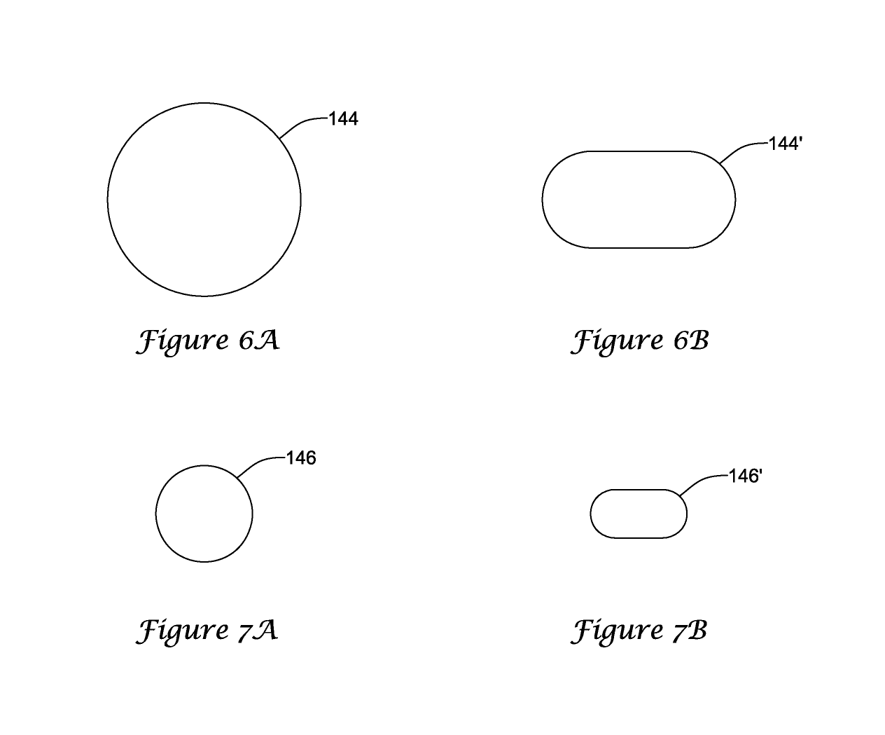

In at least some embodiments, proximal portion 144 may have a different shape, size, and/or profile than distal portion 146. For the purposes of this disclosure, differences in shape may be understood to include differences in geometric shape (e.g., circle versus oval versus square, etc.) and geometrically similar shapes of differing size. Differences in size may include differences in cross-sectional area and/or differences in cross-sectional perimeter. Differences in profile may include differences in size and/or shape. In at least some embodiments, proximal portion 144 may have a generally circular cross-sectional shape as depicted in FIG. 6A. Other shapes are contemplated. For example, proximal portion 144′ is shown in FIG. 6B with a non-circular (e.g., oval) cross-sectional shape. Distal portion 146 may have a generally circular cross-sectional shape as depicted in FIG. 7A. Other shapes are contemplated. For example, distal portion 146′ is shown in FIG. 7B with a non-circular (e.g., oval) cross-sectional shape. Numerous other shapes are contemplated for proximal portion 144 and distal portion 146 including triangular, square, rectangular, hexagonal, polygonal, regular shapes, irregular shapes, and the like.

In at least some embodiments, proximal portion 144 may have a circular cross-section shape and distal portion 146 may have a smaller circular cross-sectional shape. Alternatively, proximal portion 144 may have a non-circular cross-section shape and distal portion 146 may have a smaller non-circular cross-sectional shape. While in some embodiments the cross-sectional shape of proximal portion 144 and distal portion 146 are the same, other embodiments are contemplated where the shapes differ. For example, one of portions 144/146 may be circular and the other portion 144/146 may be non-circular.

In general, distal portion 146 may generally be reduced in size when compared to proximal portion 144. This may include reducing distal portion 146 by 1F or more relative to proximal portion 144. For example, while not intending to be limited to any particular dimensions, proximal portion 144 may have an outer diameter in the range of about 0.03 to 0.10 inches, or about 0.05 to 0.07 inches, or about 0.06 to 0.07 inches, or about 0.068 inches. Such dimension may be suitable for use with a typical guide catheter (e.g., a guide catheter having an inner diameter of about 0.07 inches or so). Distal portion 146 may have an outer diameter in the range of about 0.04 to 0.09 inches, or about 0.05 to 0.07 inches, or about 0.06 inches. Tapered portion 148 may form a gradual transition from proximal portion 144 to distal portion 146 over a length (e.g., about 1-10 cm, or about 1-5 cm, or about 2 cm). Tapered portion 148 may have a leading edge defining a taper angle. The taper angle may vary. For example, the taper angle may be in the range of about 30-85 degrees or about 45-75 degrees. These are just examples.

The relative lengths of portions 144/146 may also vary. For example, proximal portion 144, distal portion 146, or both may have a length in the range of about 1-40 cm, 2-20 cm, 5-15 cm, or about 10 cm. The relative lengths of portions 144/146 may be the same or different. The wall thickness of distal sheath 126 may also vary. For example, distal sheath 126 may have a wall thickness in the range of about 0.001 to 0.010 inches, or about 0.002 to 0.008 inches, or about 0.003 to 0.006 inches. The wall thickness along proximal portion 144 and along distal portion 146 may be the same or may be different. These are just examples.

Manufacturing distal sheath 126 may include using a mandrel having a shape corresponding to the desired shape for portions 144/146/148. For example, the mandrel may include a larger portion corresponding to proximal portion 144, a smaller portion corresponding to distal portion 146 and a taper corresponding to tapered portion 148. The manufacturing process may include conventional reflow processes or other suitable processes.

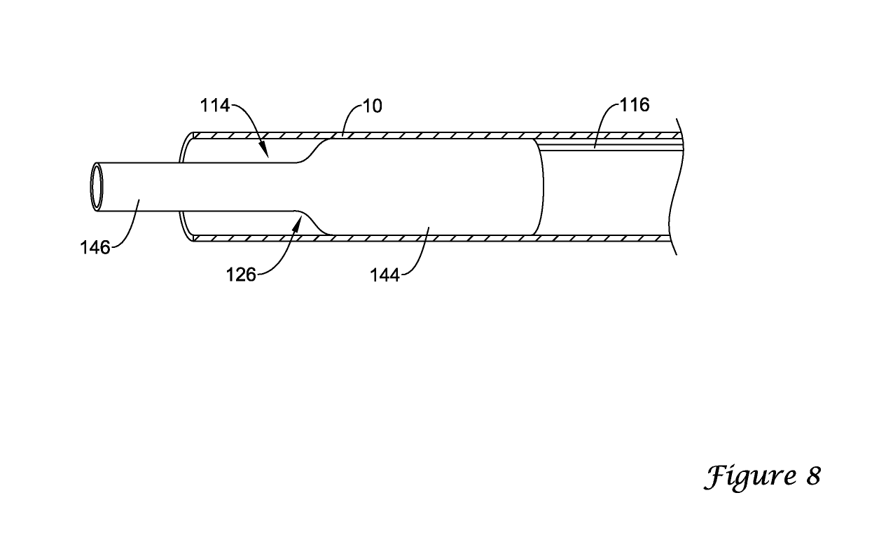

FIG. 8 illustrates guide extension catheter 114 disposed within guide catheter 10. Here it can be seen that proximal portion 144 has an outer diameter that may closely approximate the inner diameter of guide catheter 10. In addition, distal portion 146 has a reduced outer diameter when compared with proximal portion 144, while still being sufficiently sized to accommodate a therapeutic medical device therein.

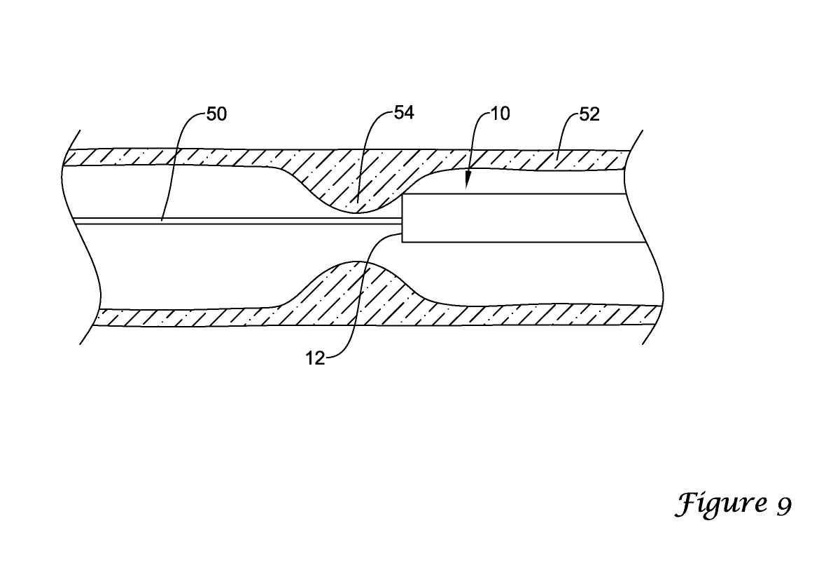

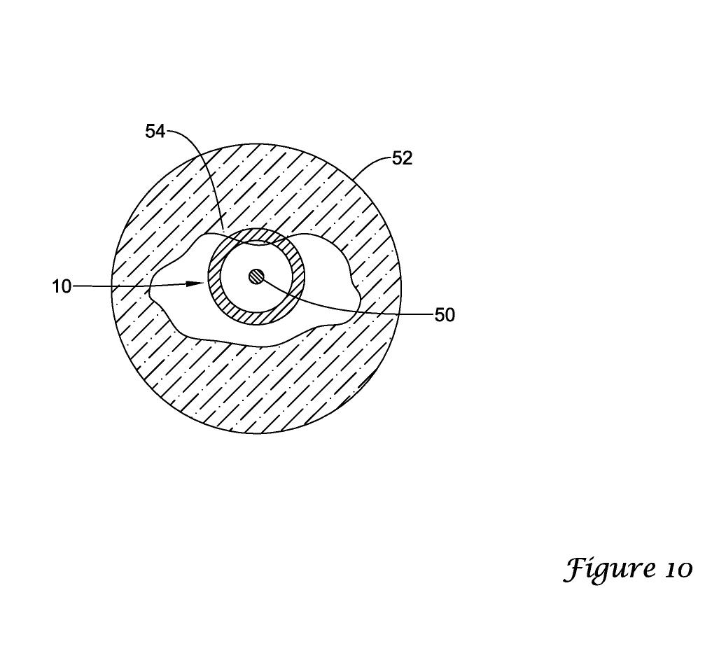

FIG. 9 illustrates guide catheter 10 advancing over a guidewire 50 through a blood vessel 52 to a position adjacent to a lesion 54. In this example, lesion 54 may at least partially occlude vessel 52. Such lesions 54 may present technical challenges for navigation therethrough by, for example, medical devices with a circular end. In particular, calcified lesions may pose challenges for the navigation of medical devices having a rounded or circular distal end. For example, distal end 12 of guide catheter 10 may have a tendency to catch on lesion 54 or otherwise be blocked from easily navigating past lesion 54 as shown in FIG. 10.

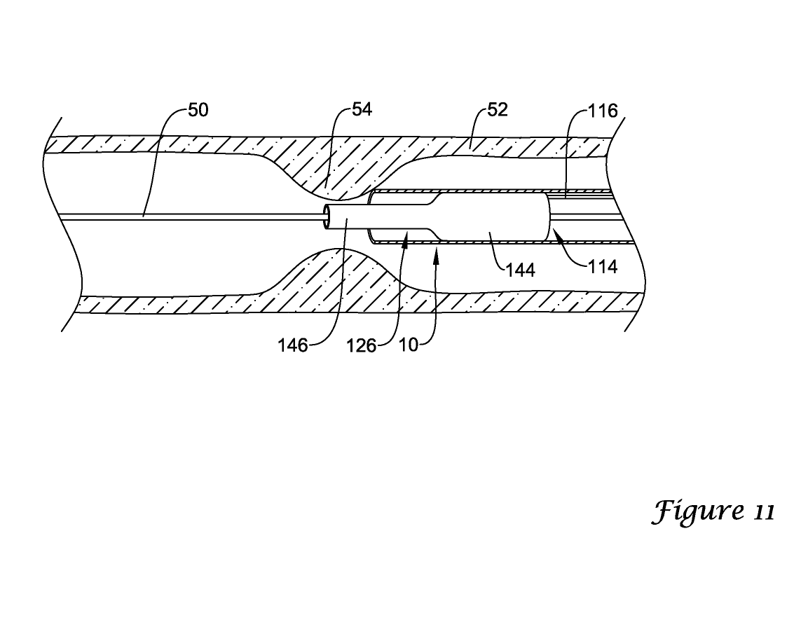

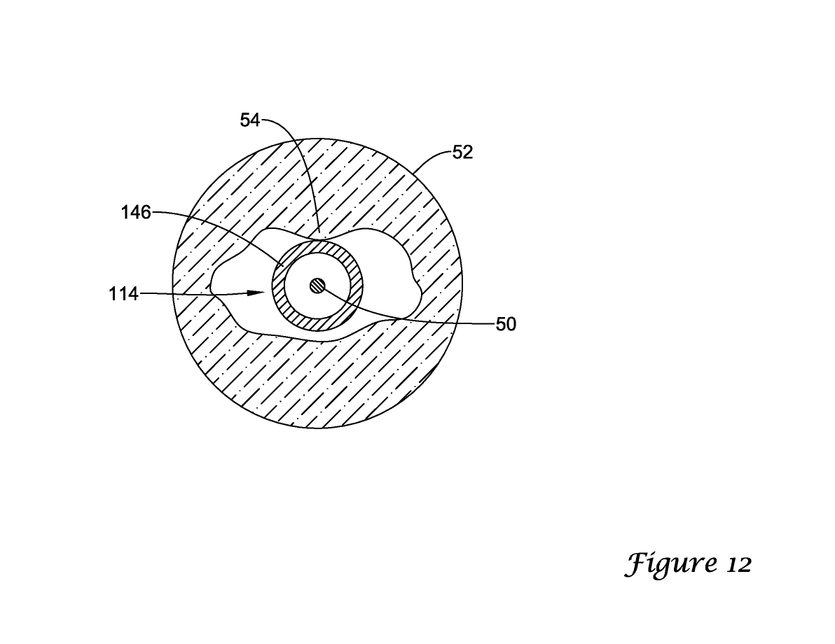

FIG. 11 illustrates guide extension catheter 114 disposed within guide catheter 10. Here it can be seen that distal portion 146 may extend distally out from guide catheter 10. This may desirably form a tapered or reduced leading edge that may aid in navigating guide catheter 10 and/or guide extension catheter 114 and/or other therapeutic medical device past lesion 54 as shown in FIG. 12.

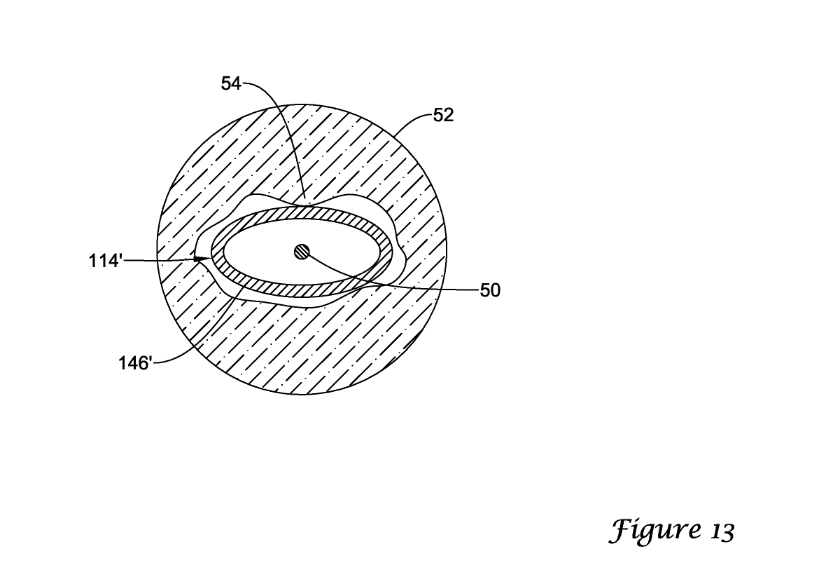

While distal portion 146 is depicted in FIG. 12 as having a generally circular cross-sectional shape, other shapes are contemplated including those shaped disclosed herein. For example, FIG. 13 illustrates guide extension catheter 114′ where distal portion 146′ has a non-circular (e.g., oval) shape. Such a shape may have at least one outer dimension that is reduced. This “flattened” distal portion 146′ may improve the ability of guide extension catheter 114′ to be advanced through lesion 54.

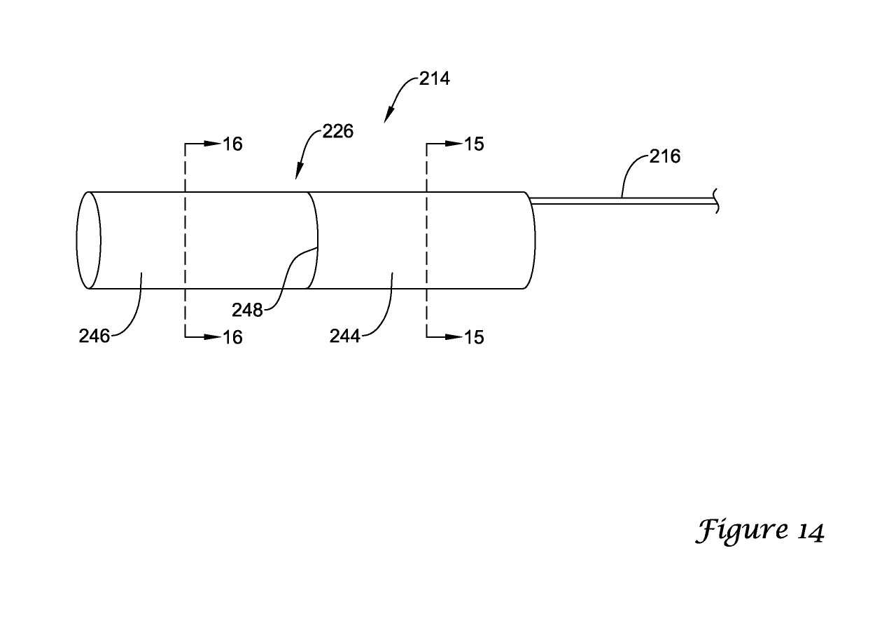

FIG. 14 illustrate an example guide extension catheter 214 that may be similar in form and function to other guide extension catheters disclosed herein. Guide extension catheter 214 may include proximal member 216 and distal sheath 226. The structures are shown schematically. It can be appreciated that the form and/or structural configuration of proximal member 216 and/or distal sheath 226 may resemble other proximal members and distal sheaths disclosed herein. Distal sheath 226 may include proximal portion 244, distal portion 246, and transition portion 248 disposed between proximal portion 244 and distal portion 246.

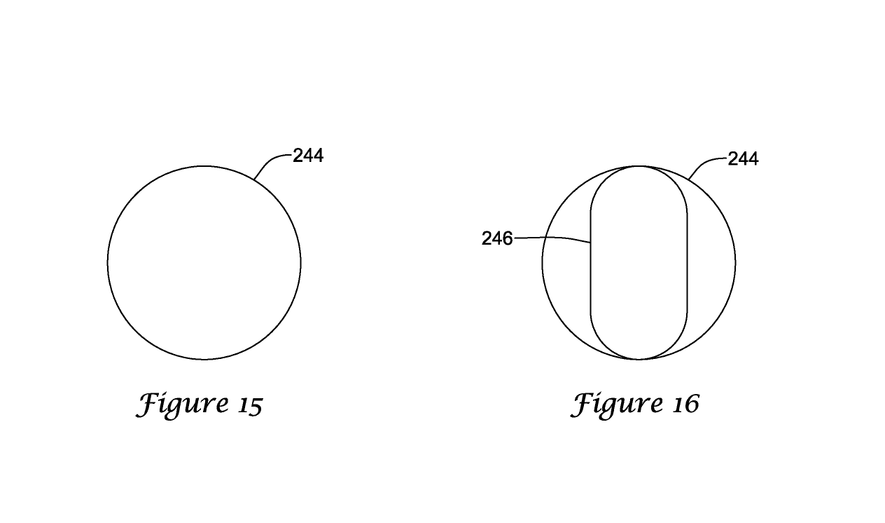

In at least some embodiments, proximal portion 244 may have a different shape, size, and/or profile than distal portion 246. For example, proximal portion 244 may have a generally circular cross-sectional shape as depicted in FIG. 15. Other shapes are contemplated. Distal portion 246 may have a generally non-circular (e.g., oval) cross-sectional shape as shown in FIG. 16. Numerous other shapes are contemplated for proximal portion 244 and distal portion 246.

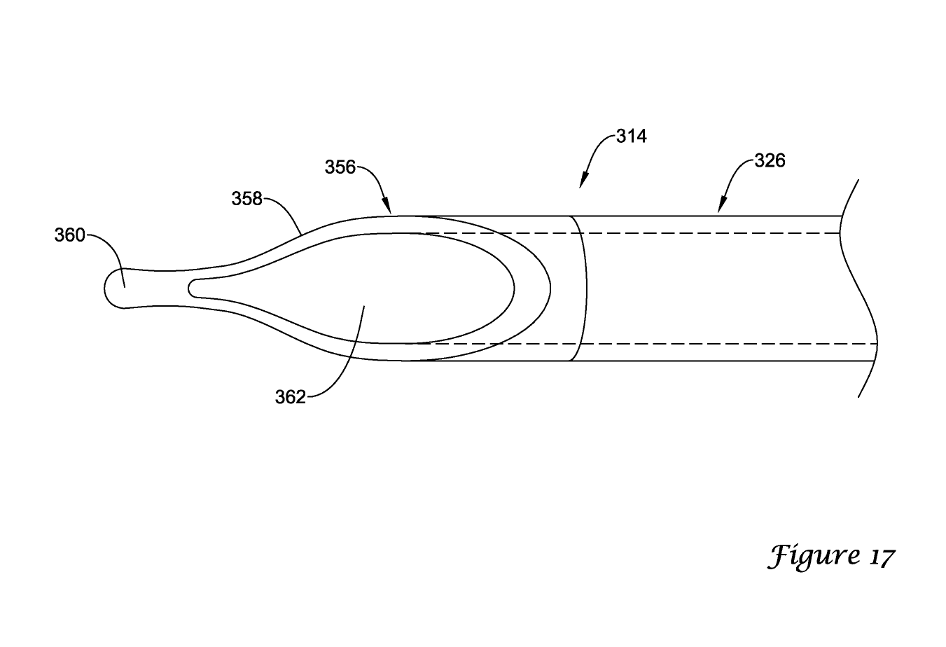

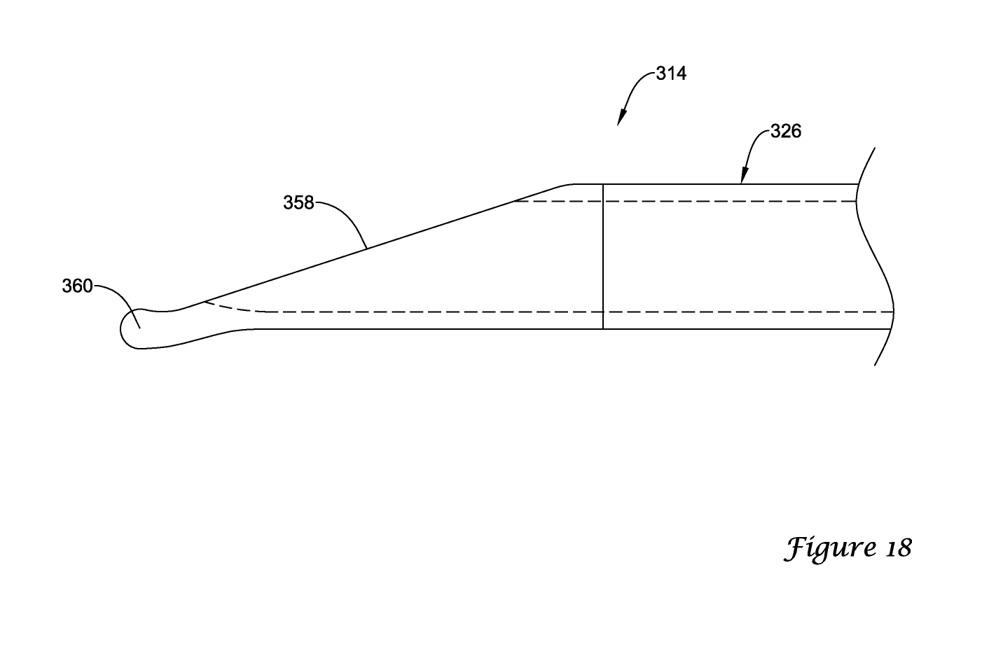

FIGS. 17-18 illustrate an example guide extension catheter 314 that may be similar in form and function to other guide extension catheters disclosed herein. Guide extension catheter 314 may include distal sheath 326 with a tip member 356. Tip member 356 may include an angled edge 358 defining an angled opening 362. Tip member 356 may also include an atraumatic lip 360. Tip member 356 may be utilized with any of the guide extension catheters disclosed herein.

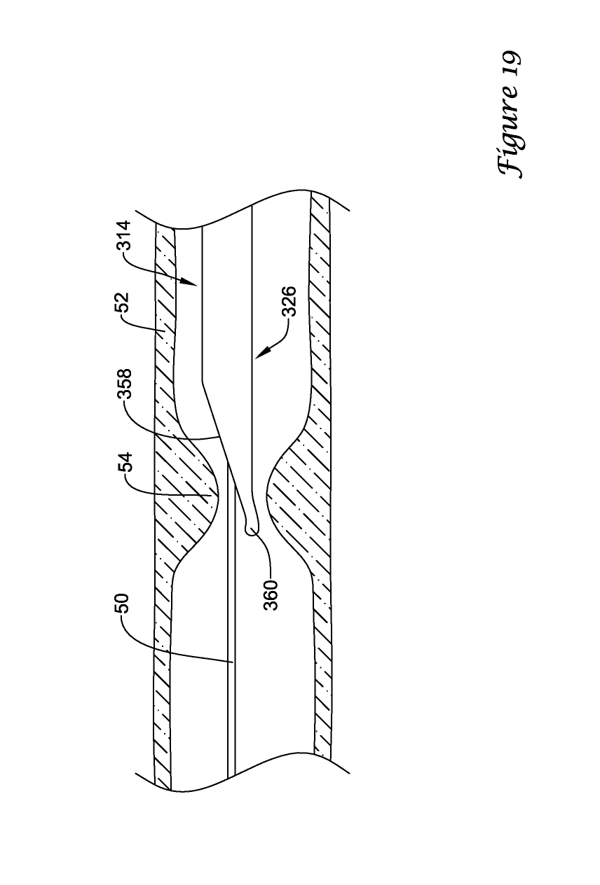

FIG. 19 illustrates guide extension catheter 114 disposed in blood vessel 52. Here it can be seen that angled edge 358 and lip 360 may aid in navigating guide extension catheter 314 (and/or guide catheter 10 and/or a therapeutic medical device) past lesion 54. While guide catheter 10 is not shown, it can be appreciated that distal sheath 326 may extend outer from distal end 12 of guide catheter 10.

The materials that can be used for the various components of the guide extension catheters disclosed herein may vary. For simplicity purposes, the following discussion makes reference to proximal member 16 and distal sheath 26. However, this is not intended to limit the devices and methods described herein, as the discussion may be applied to other similar tubular members and/or components of tubular members or devices disclosed herein.

Proximal member 16 and distal sheath 26 and/or other components of guide extension catheter 14 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of proximal member 16 and/or distal sheath 26 may also be loaded with, made of, or otherwise include a radiopaque material. Radiopaque materials are understood to be materials capable of producing a relatively bright image on a fluoroscopy screen or another imaging technique during a medical procedure. This relatively bright image aids the user of guide extension catheter 14 in determining its location. Some examples of radiopaque materials can include, but are not limited to, gold, platinum, palladium, tantalum, tungsten alloy, polymer material loaded with a radiopaque filler (e.g., barium sulfate, bismuth subcarbonate, etc.), and the like. Additionally, other radiopaque marker bands and/or coils may also be incorporated into the design of guide extension catheter 14 to achieve the same result.

In some embodiments, a degree of Magnetic Resonance Imaging (MRI) compatibility is imparted into guide extension catheter 14. For example, proximal member 16 and distal sheath 26, or portions thereof, may be made of a material that does not substantially distort the image and create substantial artifacts (i.e., gaps in the image). Certain ferromagnetic materials, for example, may not be suitable because they may create artifacts in an MRI image. Proximal member 16 and distal sheath 26, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of proximal member 16 and distal sheath 26 that may define a generally smooth outer surface for guide extension catheter 14. In other embodiments, however, such a sheath or covering may be absent from a portion of all of guide extension catheter 14, such that proximal member 16 and distal sheath 26 may form the outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the guide extension catheter 14 (including, for example, the exterior surface of proximal member 16 and distal sheath 26) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of proximal member 16 and distal sheath 26, or other portions of guide extension catheter 14. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A guide extension catheter, comprising:
a proximal member having a proximal outer diameter;
a distal sheath member attached to the proximal member, the distal sheath member having a proximal sheath portion and a distal sheath portion, the proximal sheath portion having an outer diameter greater than the proximal outer diameter;
wherein the proximal sheath portion a first cross-sectional profile; and
wherein the distal sheath portion has a second cross-sectional profile different from the first cross-sectional profile.

2. The guide extension catheter of claim 1, wherein the first cross-sectional profile, the second cross-sectional profile, or both are circular.

3. The guide extension catheter of claim 1, wherein the first cross-sectional profile, the second cross-sectional profile, or both are non-circular.

4. The guide extension catheter of claim 1, wherein the first cross-sectional profile and the second cross-sectional profile are the same shape and differ in size.

5. The guide extension catheter of claim 1, wherein the first cross-sectional profile and the second cross-sectional profile differ in shape.

6. The guide extension catheter of claim 1, wherein the distal sheath member includes a tapered region disposed between the proximal sheath portion and the distal sheath portion.

7. The guide extension catheter of claim 1, wherein the distal sheath portion includes a taper.

8. The guide extension catheter of claim 1, wherein the distal sheath portion includes a tip member.

9. The guide extension catheter of claim 8, wherein the tip member includes an angled distal opening.

10. The guide extension catheter of claim 8, wherein the tip member includes an atraumatic lip portion.

11. The guide extension catheter of claim 1, wherein the proximal member includes a hypotube.

12. A guide extension catheter system, comprising:
a guide catheter having an inner diameter; and
a guide extension catheter extending through the guide catheter, the guide extension catheter comprising:
a proximal shaft,
a distal sheath member attached to the proximal shaft, the distal sheath member having proximal portion, a distal portion, and a tapered portion disposed between the proximal portion and the distal portion, and
wherein the proximal portion of the distal sheath member has an outer diameter that is configured to substantially fit within the inner diameter of the guide catheter.

13. The guide extension catheter system of claim 12, wherein the proximal portion of the distal sheath member has a circular cross-sectional shape.

14. The guide extension catheter system of claim 13, wherein the distal portion of the distal sheath member has a non-circular cross-sectional shape.

15. The guide extension catheter system of claim 13, wherein the distal portion of the distal sheath member has a circular cross-sectional shape and wherein an outer diameter of the distal portion of the distal sheath member is smaller than the outer diameter of proximal portion of the distal sheath member.

16. The guide extension catheter system of claim 12, wherein the distal portion of the distal sheath member includes a tip member with an atraumatic lip.

17. The guide extension catheter system of claim 16, wherein the tip member includes an angled distal opening.

18. The guide extension catheter system of claim 12, wherein the proximal shaft has a lumen formed therein.

19. A method for accessing a coronary artery, the method comprising:
providing a guide catheter;
advancing the guide catheter through a blood vessel to a location adjacent to an ostium of a coronary artery;
providing a guide extension catheter, the guide extension catheter including:
a proximal shaft,
a distal sheath member attached to the proximal shaft, the distal sheath member having proximal portion, a distal portion, and a tapered portion disposed between the proximal portion and the distal portion, and
wherein the proximal portion of the distal sheath member has an outer diameter greater than an outer diameter of the proximal shaft;
advancing the guide extension catheter through the guide catheter to a position where at least a portion of the distal sheath member extends distally beyond a distal end of the guide catheter and into the coronary artery; and
advancing a treatment catheter through the guide catheter.

20. The method of claim 19, wherein the distal portion of the distal sheath member includes an atraumatic lip and an angled distal opening.
